(19) United States
(12) Patent Application Publication
(10) Pub. No.: US 2024/0415580 A1
(43) Pub. Date: Dec. 19, 2024

(54) BACK TABLE OPTIMIZATION USING REAL TIME SURGICAL ACTIVITY RECOGNITION

(71) Applicant: Kaliber Labs Inc., San Francisco, CA (US)

(72) Inventors: Ryan BARBAN, San Francisco, CA (US); Rithesh PUNYAMURTHULA, San Francisco, CA (US); Aneesh JONELAGADDA, San Francisco, CA (US); Chandra JONELAGADDA, San Francisco, CA (US)

(21) Appl. No.: 18/745,993

(22) Filed: Jun. 17, 2024

Related U.S. Application Data
(60) Provisional application No. 63/508,873, filed on Jun. 16, 2023.

(51) Int. Cl. A61B 34/20; A61B 34/00; G16H 40/20

(52) U.S. Cl. CPC A61B 34/20; A61B 34/25; G16H 40/20; A61B 2034/2065; A61B 2034/252; A61B 2034/254

(57) ABSTRACT

Methods and apparatuses (e.g., systems, including in particular software) for assisting a technician (e.g., surgeon, surgical technician, nurse, assistant, etc.) in preparing one or more tools for use during one or more surgical procedures to efficiently assist in a medical (e.g., surgical) procedure.

### CLAIM OF PRIORITY

This patent application claims priority to U.S. Provisional Patent Application No. 63/508,873, titled “BACK TABLE OPTIMIZATION USING REAL TIME SURGICAL ACTIVITY RECOGNITION,” filed on Jun. 16, 2023, and herein incorporated by reference in its entirety.

### BACKGROUND

The operating room has sterile and non-sterile environments. The sterile environment is where the surgeons, surgical assistants, and surgical technicians (e.g., scrub technicians) work. The primary function of a scrub technician in an operating room is to prepare and maintain a region called the back table in the sterile region. The scrub technician lays out the surgical instruments which the surgeon is likely to need and hands the right instrument to the surgeon at the right time.

Proper coordination between the scrub technician and the surgeon is essential to an efficient surgical procedure. Surgeries could take twice to three times as long when the coordination breaks down. Typically, the scrub technician is a nurse who supports multiple surgeons, who have their own preference of surgical tools and techniques. In addition, the presence of unanticipated pathology would initiate additional interventions, surgical tasks, which could require additional tools.

### SUMMARY OF THE DISCLOSURE

Described herein are methods and apparatuses (e.g., systems, including in particular software) for assisting a technician (e.g., surgeon, surgical technician, nurse, assistant, etc.) in preparing one or more tools for use during one or more surgical procedures to efficiently assist in a medical (e.g., surgical) procedure.

In some embodiments, the invention includes hardware, software and/or firmware, including configured to perform the automated or semi-automated procedure including: processing video input from one or more surgical fields of view in real-time (e.g., forming one or more video streams); optionally, processing the video input from one or more cameras imaging (e.g., placed over) the back table (forming one or more video streams); analyzing the video streams using deep learning and computer vision techniques in real-time; and recognizing the surgical actions and the surgical context, i.e., which surgical procedure/sub-procedure in which these actions take place. The surgical context may include an overall situational awareness, which may include recognizing the anatomy and/or the pathology encountered in the surgical field of view, preceding actions, the patient's unique medical history, etc. In general, these methods and apparatuses may include anticipating the upcoming surgical sub-procedure(s) being performed. In some examples these methods and apparatuses may include mapping the upcoming sub-procedure into a specific sequence of surgical actions; the sequence may be based on a predetermined schedule (e.g., corresponding to the procedure(s) being performed), and/or may be based on the surgeon's preferences. These methods and apparatuses may be configured to anticipate the surgical tools which will be needed by the surgeon at specific points in time based on recognizing the surgical context in the field of view.

In any of these examples the methods and apparatuses described herein may instruct the scrub technician: (1) to provide (e.g., to lay out) one or more sets of tools which would be needed for a specific surgical sub-procedure; (2) to hand a specific tool/implant to the surgeon at one or more specific points of time; (3) optionally, the method or apparatus performing the method may also recognize the layout of the instruments on the back-table, by analyzing the video feed from an external camera overlooking the back table and may confirms that the layout of the instruments matches the anticipated surgical activity subject to the surgeon's preference of techniques and tool; and (4) the method and apparatus may inform the scrub technician to alter the tool needs of the surgeon due to emergent situations in the field of view. The tool may be altered by switching out, adding or omitting a particular tool or tools. For example, the if the method or apparatus performing the method senses an excessive bleeding in the field of view, it may prompt the surgeon to pause the current surgical activity, reach for a cauterizer to seal the wound before resuming the task at hand.

For example, a method of providing surgical guidance to a scrub technician during a surgical procedure may include: identifying, using a real-time surgical context recognition module, one or more surgical procedure being performed on a patient in a sterile field, wherein the real-time surgical context recognition module receives one or more video streams of the surgical procedure being performed and one or more video streams of a back table within the sterile field; determining, using a back table instruction processor module including a trained machine learning agent, a sequence of surgical tools that will be needed to perform the identified one or more surgical procedures; outputting, to a monitor visible within the sterile field, each of the surgical tools within the sequence, wherein the surgical tools are presented sequentially for arrangement on the back table within the sterile field; receiving input from a back table camera viewing the back table; and verifying, by the back table instruction processor module, that the surgical tools within the sequence have been provided on the back table.

Also described herein are systems of performing any of these methods. In general, these systems may include one or more processors and a memory coupled to the one or more processors. The memory may hold computer-program instructions, that, when executed by the one or more processors, perform the methods. For example, the memory may hold computer-program instructions, that, when executed by the one or more processors, perform the method of: identifying, using a real-time surgical context recognition module, one or more surgical procedure being performed on a patient in a sterile field, wherein the real-time surgical context recognition module receives one or more video streams of the surgical procedure being performed and one or more video streams of a back table within the sterile field; determining, using a back table instruction processor including a trained machine learning agent, a sequence of surgical tools that will be needed to perform the identified one or more surgical procedures; outputting, to a monitor visible within the sterile field, each of the surgical tools within the sequence, wherein the surgical tools are presented sequentially for arrangement on the back table within the sterile field.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates one example of an apparatus as described herein.

FIG. 2 illustrates an example of a visual output for a scrub technician.

FIG. 3 illustrates an example of a visual output for a scrub technician.

FIG. 4 illustrates an example of a visual output for a scrub technician.

### DETAILED DESCRIPTION

Surgical activity often involves the performance of specific actions using certain implements on a given set of anatomical structures in specific regions. In this sense, a surgical activity may be described as a more abstract concept compared to surgical tasks. Sometimes, the activity can span several individual surgical actions and in some cases, the activity could involve fewer individual steps. The methods and apparatuses may refer to the abstract form of a surgical activity which includes the surgical procedure comprising a composition of several activities performed in a given sequence.

For example, the methods and apparatuses described herein may include a real-time Surgical Context Recognition (SCR) module and a Back Table Instruction Processor (BTIP), each of which are described in greater detail here. In general these method and apparatuses, and in particular the real-time surgical context recognition module and the back table instruction processor, may address problems that have were difficult or impossible to successfully resolve using existing techniques. The CR and BTIP may be part of a single system or sub-system. These methods and apparatuses may also allow the rapid and effective (e.g., in real time) assistance during a surgical procedure by more efficiently processing data from one or more video streams, extract essential information from the correct video streams, and provide actionable information or output to assist in an (often time-sensitive) surgical procedure.

### Real Time Surgical Context Recognition Module

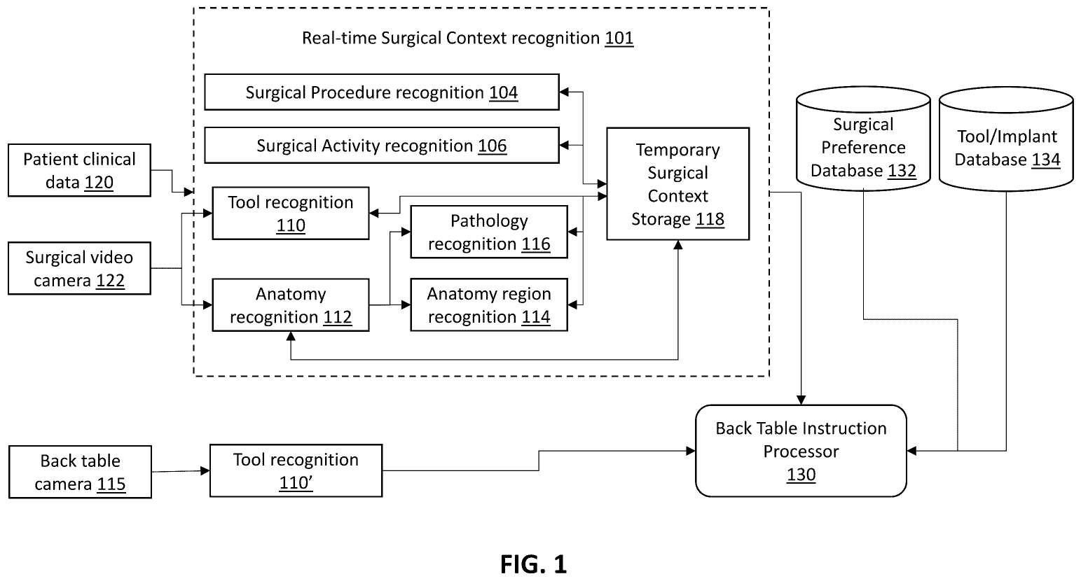

The real time surgical context recognition module, also referred to herein as an SCR (or real time SCR), may be implemented using a real-time video processing system, which may use machine learning (e.g., artificial intelligence, AI). The SCR may also be referred to as a system for providing back table instruction, e.g., to a medical support staff, such as a nurse or technician, and specifically, a scrub technician. This system may be part of or used with another surgical assistance/guidance system or subsystem that may orchestrate the video stream and data extracted from it between several sub-modules which perform specific tasks. These sub-modules may be implemented using machine learning (e.g., deep learning) techniques and computer vision techniques, modified and implemented as described herein. The SCR may also use a temporary internal storage, labeled as Temporary Surgical Context Storage (TSC). FIG. 1 illustrates one example of a schematic of a system (e.g., an SCR) including a TSC. In the illustration of FIG. 1, the SCR, including the TSC, is configured to recognize surgical activity and surgical procedures and interpret them in a context that can provide meaningful and useful context (output) to the medical support staff.

In FIG. 1, the system is schematically shown as a plurality of interconnected modules that may provide the direct signals, i.e., output into the TSC and out of the system. In this example, the system (the real time SCR system 101) may include an anatomy recognition module 112; a pathology recognition module 116; an anatomy region recognition module 114; and a tool recognition module 110. These modules may utilize the context provided by either the inputs (e.g., surgical video input 122 and/or patient clinical data 120) and/or the output of the temporary surgical context storage module (TSC) 118, to boost the confidence of the recognition tasks they perform. For instance, in the case of the tool recognition module 110, the TSC may be used as illustrated below.

For example, as the surgeon manipulates one or more surgical tools, the endoscopic camera (providing surgical video camera input 122) may temporarily lose sight of the tool or may sight a non-identifiable part of the tool. The tool recognition module 110 may use the TSC 118 to make its final prediction by leveraging the temporary storage and heuristically determine if a tool could have been changed since the last time it was recognized confidently. The tool recognition module may apply a variety of techniques, including object tracking, hue changes in the tool, smoothing the output over time, etc., to boost the confidence of the recognition.

Similarly, the anatomy 112, pathology 116, and anatomy region 114 recognition modules may also leverage the context to disambiguate structures by leveraging the context, for example, field of view in the preceding seconds provides information about where the camera is currently pointing.

Higher-level modules may utilize the context provided by the TSC to perform their tasks.

### Surgical Activity Recognition

In some cases the machine learning agent, e.g., for recognizing surgical activity, may be configured as a transformer network. One or more transformer-networks (e.g., as one example of a type of deep learning technique that may be used herein), may be used to output a sequence of tokens based on a sequence of input tokens. Examples may include transformers used for language translation and sentence completion tasks. Transformers have also been used to recognize sports activity from video streams. The methods and apparatuses described herein may extend a specific variation of the transformer network, referred to herein as videoBERT, which stands for video bi-directional transformers. In a general sense, these networks may be trained to output text description of video streams, they model the joint distribution of text and video data.

The methods and apparatuses described herein may be an improvement over traditional implementations of transformer-based networks, which may be used for surgical activity recognition that may otherwise have failed in the surgical domain. Described herein is an approach to the use of transformer-based surgical activity recognition that may leverage the real-time tool, anatomy, pathology, and anatomy region recognition models. These methods and apparatuses may use feature extraction performed on the output of these models and the feature vectors may be assembled for each frame in the input video stream. In some examples, subject matter experts may provide extensive textual descriptions of various activities, tools, anatomy and pathology seen in the video stream (e.g., for training). These textual descriptions and features may be extracted from the video stream, as described above, and may be used to construct joint distributions over sequences of feature matrices and surgical semantic tokens produced by subject matter experts.

### Surgical Procedure Recognition

The methods and apparatuses described herein may also recognize a more abstract form of surgical activity which is the surgical procedure, which is a composition of several activities performed in a given sequence. Thus, any of these systems may include a surgical activity recognition module 106. The surgical recognition module may include a trained machine learning agent that may recognize the surgical activity being performed, e.g., from the video input 122 and/or from the patient clinical data.

### Back Table Instruction Processor

Any of these methods and apparatuses may include a back table instruction processor 130 that may receive in input from the real-time surgical context recognition module 101 as well as the specific back table camera 115, which may separately use the same or a different iteration of the tool recognition module 110′. The back table instruction processor may also access one or more databases, including a surgical preference database 132 and/or a tool/implant database 134. The back table instruction processor may synthesize this information in order to prepare instructions, via an output (e.g., graphical and/or text output) for display to the medical support staff, to intelligently provide with advance instruction on which tools the surgeon(s) may need based on the SCR output, and may verify (e.g., from the back table camera/video input as well as the tool recognition module 110′) the correct tool is being provided/prepared within the sterile field. The back table instruction processor may also provide context-specific and appropriate textual output coordinated with the physicians (surgeons) performing the procedure.

In general, this output may be provided to the medical support staff (e.g., scrub technician) within the sterile field on a dedicated display. This display (user interface) may be interactive, or non-interactive, with the medical support staff. The output may be responsive to both the ongoing surgical procedure, in real time, as well as the actions by the medical support staff on the back table region, in preparing the tools for access by the surgeon(s).

These systems (e.g., the back table instruction processor module, BTIP) may be dynamic and may respond to changes in the procedure based on the SCR module and/or the back table camera and tool recognition module.

Thus, once the surgical activity is recognized in real time, the BTIP module may translate this into specific instructions to the scrub technicians. This module may decouple surgeon preferences, i.e., specific tools and techniques used by a given surgeon, and the recognition of the activity itself. In this manner, the system can be easily configured to meet the needs of different surgeons by merely changing the surgeon preference and tool/implant databases. The BTIP may map the surgical activity predicted by the SAR module into a sequence of tools needed by the surgeon.

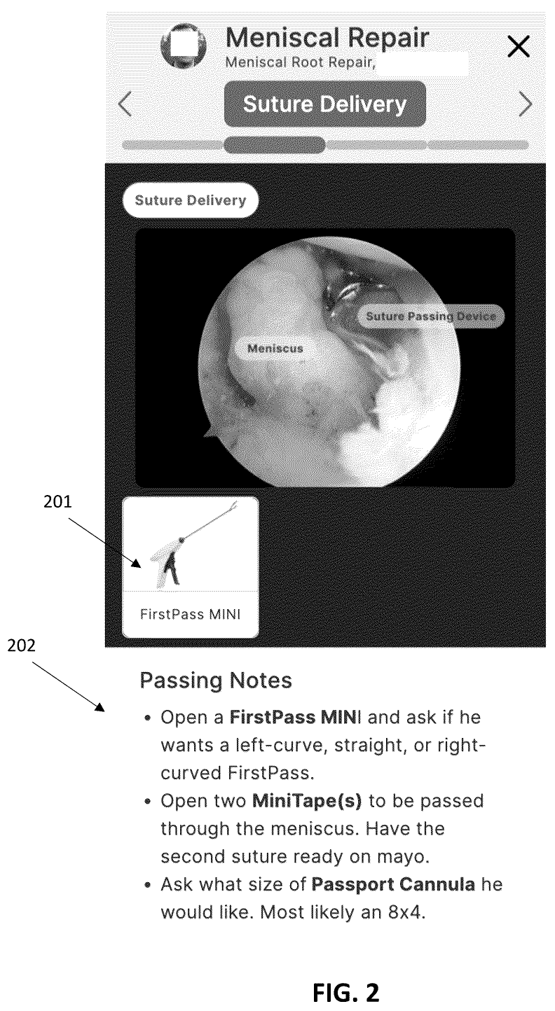

For example, FIGS. 2-4 show examples of outputs that may be provided by the back table instruction processor module 130 as described herein. FIGS. 2-4 show screenshots from the output of the back table instruction processor, illustrating how the support technician can assist the surgeon. For example, the system including the back table instruction processor module may recognize surgical activities (e.g., “Suture Delivery”, “Femoral Drilling Preparation”, etc.) from the real time surgical context recognition module 101 within a corresponding surgery.

In some cases the back table instruction processor may use a trained machine learning agent to synthesize the input from the real time SCR 101 and the back table camera (and/or tool recognition module 110′), and in some cases the surgical preference database 132 and/or tool/implant database 134, and may provide output. For example the system may be configured to display video and/or image highlights of the surgery in the application, and/or apply helpful anatomical labels, as shown. In some cases the system may display the live video stream of the surgery in the application, automatic labeling on the live stream.

As shown in FIG. 2, the system and/or method may display the current and/or predicted next step in the procedure, e.g., suturing, suture delivery. The system and/or method may then identify which tool(s) 201 to provide (e.g., “FirstPass Mini” suture passer). In FIG. 2 the output include an image of the procedure (or a similar procedure) and may include notes (“passing notes” 202) with doctor and/or facility preferences for the attention of the scrub technician preparing the tool on the back table, including prompts to remind the scrub technician how to prepare the tool and/or what to ask the surgeon to assist in preparing the tool(s).

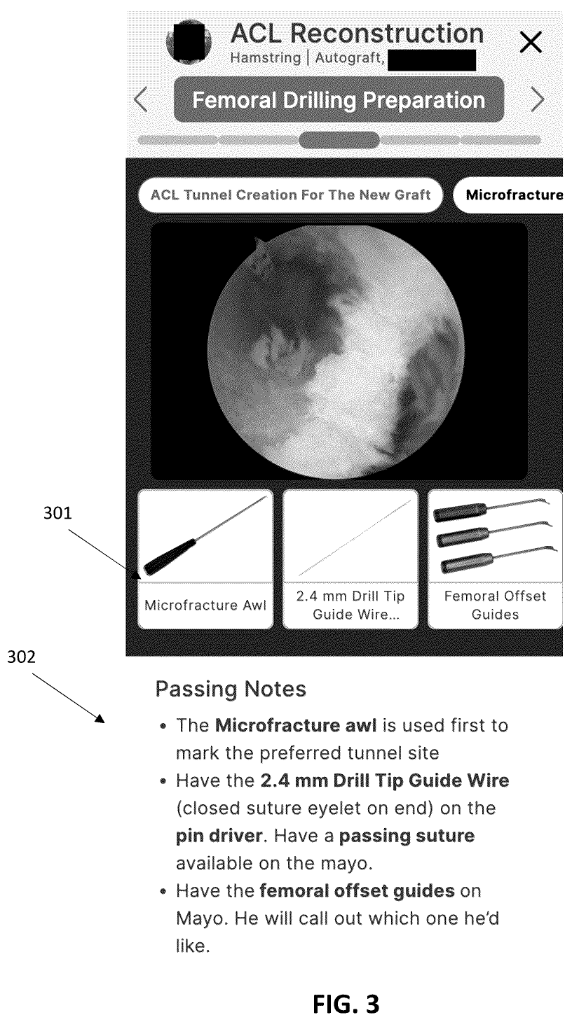

FIG. 3 shows another example, showing tools to be prepared 301 and notes 302 during an identified stage of a procedure (femoral drilling preparation). The system may detect the procedure (e.g., ACL reconstruction) and may identify the stage and determine what tools the surgeon is going to need based on the input from the surgical context recognition module 101 and/or databases.

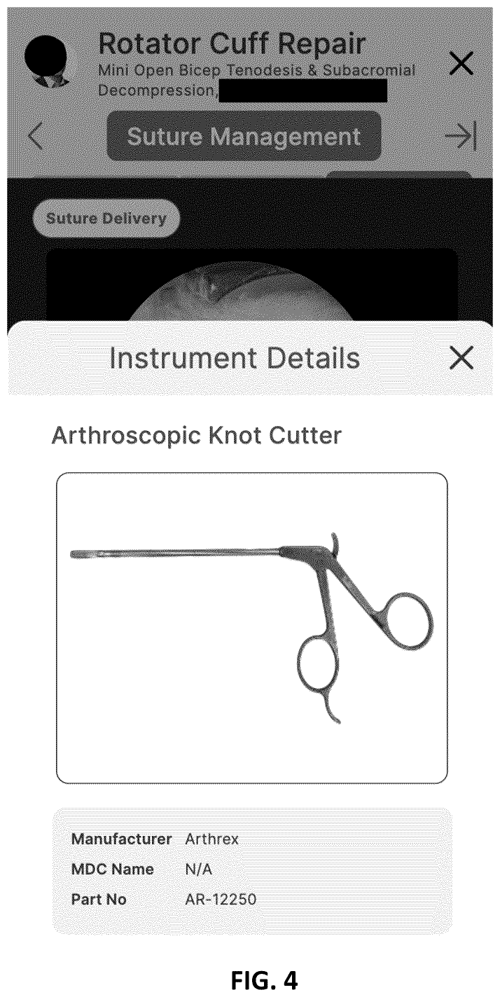

These systems may also confirm that the proper tool has been prepared, e.g., using the input from the back table camera and tool recognition module, as described above. In some cases the system may provide additional information and/or detail to recognize the tools being provided, as shown in FIG. 4. In this example the user (e.g., scrub technician) may request additional details on the instrument to be provided (e.g., an arthroscopic knot cutter) as part of the procedure.

### Optional Enhancement

As mentioned above, the BTIP module may optionally include feedback to the medical support staff (e.g., scrub technician) that the tools prepared are correct and/or are properly prepared. For example, in some cases the BTIP module may operate on a second video stream (e.g., a back table video stream) to provide an additional level of guidance to the scrub technicians. Such an arrangement is shown in the illustration of FIG. 1. A tool recognition module may be specifically trained to recognize static tools against a suitably neutral background of the back table, and may be used to process the video camera corresponding to the back table video which views the back table. The primary output of the BTIP module may be analyzed in the context of the tools, recognized by the BTIP, on the back table. A more precise and visual feedback can be provided to the scrub technician in this manner.

As mentioned, the various modules described herein may include one or more machine learning agents for analyzing, in real time, the surgical video input 122 and/or back table video input. For example, the real time SCR module 101, the back table instruction processor module 130, the tool recognition module(s) 110, 110′, the pathology recognition module 116, the anatomy recognition module 108, and/or the anatomy region recognition module 114 may use one or more trained machine learning agent or agents. In some cases a single trained machine learning agent may be used for multiple modules. These trained machine learning agents may be distinct types, or may be similar, and may be trained on the same training data or different training data.

Any appropriate type of machine learning may be used with the methods and apparatuses described herein, including, but not limited to: supervised Machine Learning, unsupervised machine learning, semi-supervised machine learning, and reinforcement learning. In supervised learning, the machine learning agent (e.g., model) may be trained on a labelled dataset. Labelled datasets have both input and output parameters. In supervised learning algorithms learn to map points between inputs and correct outputs. It has both training and validation datasets labelled. For example, video image of annotated surgical procedures, including patient clinical data, in which all of some of the tools, anatomy and pathology are indicated, may be used to train the machine learning model, e.g., for one or more of the modules described herein (e.g., tool recognition 110, anatomy recognition 112, pathology recognition 116, anatomy region recognition 114, surgical procedure recognition 104, surgical activity recognition 106, etc.).

The trained machine learning agent may an artificial intelligence agent. The machine learning agent may be a deep learning agent. In some examples, the trained pattern matching agent may be trained neural network. Any appropriate type of neural network may be used, including generative neural networks. The neural network may be one or more of: perceptron, feed forward neural network, multilayer perceptron, convolutional neural network, radial basis functional neural network, recurrent neural network, long short-term memory (LSTM), sequence to sequence model, modular neural network, etc.

In some cases the machine learning agent may be Supervised-learning may include building an image classifier to differentiate between various surgical tools. Datasets of various surgical tools may be used to train the machine learning agent to identify and/or classify between surgical tools from these labeled images (video). Categories of supervised learning may include classification and regression. Classification deals with predicting categorical target variables, which represent discrete classes or labels. Classification algorithms learn to map the input features to one of the predefined classes, and may include one or more of: Logistic Regression, support Vector Machine, random Forest, decision Tree, K-Nearest Neighbors (KNN), Naive Bayes, etc. Regression deals with predicting continuous target variables, which represent numerical values. Regression may include linear Regression, Polynomial Regression, Ridge Regression, Lasso Regression, decision tree and random Forest regression.

Unsupervised Machine Learning is a type of machine learning technique in which a machine learning agent discovers patterns and relationships using unlabeled data. Unlike supervised learning, unsupervised learning doesn't involve providing the algorithm with labeled target outputs. The primary goal of Unsupervised learning is often to discover hidden patterns, similarities, or clusters within the data, which can then be used for various purposes, such as data exploration, visualization, dimensionality reduction, and more. There are two main categories of unsupervised learning: clustering and association. Clustering is the process of grouping data points into clusters based on their similarity. This technique is useful for identifying patterns and relationships in data without the need for labeled examples. Examples of clustering algorithms that may be used may include: K-Means Clustering algorithm, Mean-shift algorithm, DBSCAN Algorithm, Principal Component Analysis, and Independent Component Analysis. Association rule learning is a technique for discovering relationships between items in a dataset. It may identify rules that indicate the presence of one item implies the presence of another item with a specific probability. Specific types of association rule learning algorithms may include: A priori Algorithm, Eclat, and FP-growth Algorithms.

Any of the machine learning agents described herein may also or alternatively be Semi-Supervised learning agents. Semi-Supervised learning is a machine learning technique that works between the supervised and unsupervised learning and may use both labelled and unlabeled data. It's particularly useful when obtaining labeled data is costly, time-consuming, or resource-intensive. This approach is useful when the dataset is expensive and time-consuming. Semi-supervised learning is chosen when labeled data requires skills and relevant resources in order to train or learn from it. In some cases the methods and apparatuses may use semi-supervised learning in were, e.g., the image (video) training data set is not fully labeled. There are a number of different semi-supervised learning methods that may be used, including: graph-based semi-supervised learning, label propagation, co-training, self-training, and generative adversarial networks (GANs).

Any of the machine learning agents described herein may also or alternatively be configured for reinforcement Machine Learning. Reinforcement machine learning algorithm is a learning method that interacts with the environment by producing actions and discovering errors. Examples of reinforcement learning techniques that may be used include: Q-learning, SARSA (State-Action-Reward-State-Action), and Deep Q-learning.

Any of these methods and apparatuses may include a processor. A processor includes hardware that runs the computer program code. Specifically, the term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

The modules of the systems and methods described herein may include one or more engines and datastores. A computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of providing surgical guidance to a scrub technician during a surgical procedure, the method comprising:
identifying, using a real-time surgical context recognition module, one or more surgical procedure being performed on a patient in a sterile field, wherein the real-time surgical context recognition module receives one or more video streams of the surgical procedure being performed and one or more video streams of a back table within the sterile field;
determining, using a back table instruction processor including a trained machine learning agent, a sequence of surgical tools that will be needed to perform the identified one or more surgical procedures;
outputting, to a monitor visible within the sterile field, each of the surgical tools within the sequence, wherein the surgical tools are presented sequentially for arrangement on the back table within the sterile field.

2. The method of claim 1, wherein the one or more procedures comprises two or more procedures sharing the same back table within the sterile field.

3. The method of claim 1, wherein the one or more procedures comprises a single procedure.

4. The method of claim 1, wherein outputting comprises outputting the surgical tools sequentially in a time in a timed manner during the course of the one or more surgical procedures.

5. The method of claim 1, wherein the trained machine learning agent is trained to identify the sequence of surgical tools based on a doctor preference.

6. The method of claim 1, wherein identifying the one or more surgical procedure being performed comprises using a second trained machine learning agent.

7. The method of claim 1, wherein identifying and determining are performed in real time.

8. The method of claim 1, wherein identifying, using the real-time surgical context recognition module, comprises identifying one or more tools already being used by the surgical procedure.

9. The method of claim 8, wherein determining, using the back table instruction processor, comprises identifying one or more tools present on the back table.

10. The method of claim 9, further comprising using a tool recognition module.

11. The method of claim 1, wherein identifying, using the real-time surgical context recognition module, comprises receiving patient clinical data in addition to the one or more video streams of the back table within the sterile field and the one or more video streams of the surgical procedure.

12. A method of providing surgical guidance to a scrub technician during a surgical procedure, the method comprising:
identifying, using a real-time surgical context recognition module, one or more surgical procedure being performed on a patient in a sterile field, wherein the real-time surgical context recognition module receives one or more video streams of the surgical procedure being performed and one or more video streams of a back table within the sterile field;
determining, using a back table instruction processor module including a trained machine learning agent, a sequence of surgical tools that will be needed to perform the identified one or more surgical procedures;
outputting, to a monitor visible within the sterile field, each of the surgical tools within the sequence, wherein the surgical tools are presented sequentially for arrangement on the back table within the sterile field;
receiving input from a back table camera viewing the back table; and
verifying, by the back table instruction processor module, that the surgical tools within the sequence have been provided on the back table.

13. A system comprising:
one or more processors;
a memory coupled to the one or more processors, the memory storing computer-program instructions, that, when executed by the one or more processors, perform a computer-implemented method comprising:
identifying, using a real-time surgical context recognition module, one or more surgical procedure being performed on a patient in a sterile field, wherein the real-time surgical context recognition module receives one or more video streams of the surgical procedure being performed and one or more video streams of a back table within the sterile field;
determining, using a back table instruction processor including a trained machine learning agent, a sequence of surgical tools that will be needed to perform the identified one or more surgical procedures;
outputting, to a monitor visible within the sterile field, each of the surgical tools within the sequence, wherein the surgical tools are presented sequentially for arrangement on the back table within the sterile field.

14. The system of claim 13, wherein outputting comprises outputting the surgical tools sequentially in a time in a timed manner during the course of the one or more surgical procedures.

15. The system of claim 13, wherein the trained machine learning agent is trained to identify the sequence of surgical tools based on a doctor preference.

16. The system of claim 13, wherein identifying the one or more surgical procedure being performed comprises using a second trained machine learning agent.

17. The system of claim 13, wherein identifying and determining are performed in real time.

18. The system of claim 13, wherein identifying, using the real-time surgical context recognition module, comprises identifying one or more tools already being used by the surgical procedure.

19. The system of claim 18, wherein determining, using the back table instruction processor, comprises identifying one or more tools present on the back table.

20. The system of claim 19, further comprising using a tool recognition module.

21. The system of claim 13, wherein identifying, using the real-time surgical context recognition module, comprises receiving patient clinical data in addition to the one or more video streams of the back table within the sterile field and the one or more video streams of the surgical procedure.
